(19) United States
(12) Patent Application Publication
(10) Pub. No.: US 2025/0115873 A1
(43) Pub. Date: Apr. 10, 2025

(54) METHOD FOR PRODUCING HUMAN DEDIFFERENTIATED FAT CELLS AND CULTURE MEDIUM FOR PRODUCING HUMAN DEDIFFERENTIATED FAT CELLS FROM HUMAN MATURE ADIPOCYTES

(71) Applicant: Taro MATSUMOTO, Musashino-shi, Tokyo (JP)

(72) Inventors: Taro MATSUMOTO, Musashino-shi (JP); Tomohiko KAZAMA, Tokyo (JP); Yuki NAGAOKA, Tokyo (JP)

(21) Appl. No.: 18/851,111

(22) PCT Filed: Mar. 29, 2023
(86) PCT No.: PCT/JP2023/012925

§ 371 Date: Sep. 26, 2024

(30) Foreign Application Priority Data
Mar. 29, 2022 (JP) 2022-052876

(51) Int. Cl. C12N 5/077

(52) U.S. Cl. CPC C12N 5/0653; C12N 2506/13

(57) ABSTRACT

A method for producing human dedifferentiated fat cells includes a step of culturing human mature adipocytes using a culture medium containing a human platelet lysate. A culture medium for producing human dedifferentiated fat cells from human mature adipocytes includes a human platelet lysate.

### TECHNICAL FIELD

The present invention relates to a method for producing human dedifferentiated fat cells and a culture medium for producing human dedifferentiated fat cells from human mature adipocytes. Further, the present invention relates to a composition containing human dedifferentiated fat cells obtained by the method for producing human dedifferentiated fat cells.

Priority is claimed on Japanese Patent Application No. 2022-052876, filed Mar. 29, 2022, the content of which is incorporated herein by reference.

### BACKGROUND ART

Dedifferentiated fat cells (hereinafter, also referred to as “DFAT cells”) are known as cells having the same differentiation ability as mesenchymal stem cells (MSCs). The DFAT cells are cells obtained by dedifferentiating mature adipocytes and have multipotency. As a method for producing the DFAT cells, a method of isolating mature adipocytes constituting the adipose tissues and performing a ceiling culture method on the mature adipocytes has been reported (for example, see Patent Documents 1 and 2). The DFAT cells can be easily and massively produced using mature adipocytes isolated from a small amount of suctioned adipose tissues as a raw material.

Meanwhile, in a case where the cells are used for clinical applications, from the viewpoint of safety, it is desirable that a culture medium and a cell preparation reagent, which are used for culturing the cells, do not contain components derived from animals other than humans, that is, so-called “xeno-free”.

### CITATION LIST

### Patent Documents

Patent Document 1: Japanese Patent No. 5055613
Patent Document 2: Japanese Patent No. 5991687

### SUMMARY OF INVENTION

### Technical Problem

In the method for preparing DFAT cells in the related art, a culture medium containing fetal bovine serum (hereinafter, also referred to as FBS) is used in order to obtain DFAT cells by dedifferentiating mature adipocytes through ceiling culture. Therefore, in a case of packaging a cell preparation (final product) consisting of DFAT cells, it is necessary to sufficiently perform a cleaning operation to remove FBS. There is a problem that the number of cells in the final product is significantly reduced by this cleaning operation. In addition, even in a case where a cleaning operation is sufficiently performed, FBS in the final product cannot be completely removed, and there is an issue in transplantation safety, such as being unable to be administered to a patient having an allergy to bovine protein.

Therefore, an object of the present invention is to provide a method for producing human DFAT cells, which enables production of human DFAT cells from human mature adipocytes without using FBS, and a culture medium for producing human DFAT cells from human mature adipocytes and a composition obtained by the method for producing human DFAT cells, which are used in the method for producing human DFAT cells.

### Solution to Problem

The present disclosure includes the following aspects.
(1) A method for producing human dedifferentiated fat cells, including: a step of culturing human mature adipocytes using a culture medium containing a human platelet lysate (hereinafter, also referred to as “HPL”).
(2) The method for producing human dedifferentiated fat cells according to (1), in which a concentration of the HPL in the culture medium is 0.5 w/v % or greater and 20.0 w/v % or less.
(3) The method for producing human dedifferentiated fat cells according to (2), in which the concentration of the HPL in the culture medium is 5.0 w/v % or greater and 15.0 w/v % or less.
(4) The method for producing human dedifferentiated fat cells according to any one of (1) to (3), in which the human mature adipocytes are cultured by ceiling culture.
(5) The method for producing human dedifferentiated fat cells according to any one of (1) to (4), in which the culture medium is a culture medium that does not contain a component derived from an animal other than a human.
(6) A method for producing human dedifferentiated fat cells, including: a step of culturing human dedifferentiated fat cells using a culture medium containing HPL.
(7) The method for producing human dedifferentiated fat cells according to (6) in which a concentration of the HPL in the culture medium is 0.5 w/v % or greater and 20.0 w/v % or less.
(8) The method for producing human dedifferentiated fat cells according to (6) or (7), in which the human dedifferentiated fat cells are cultured by adhesion culture.
(9) A culture medium for producing human dedifferentiated fat cells from human mature adipocytes, the culture medium including: HPL.
(10) The culture medium for producing human dedifferentiated fat cells from human mature adipocytes according to (9), in which the culture medium does not contain a component derived from an animal other than a human.
(11) A composition including: human dedifferentiated fat cells; and HPL.

### Advantageous Effects of Invention

According to the present invention, it is possible to provide a method for producing human DFAT cells, which enables production of human DFAT cells without using FBS, and a culture medium for producing human DFAT cells from human mature adipocytes and a composition obtained by the method for producing human DFAT cells, which are used in the method for producing human DFAT cells.

### BRIEF DESCRIPTION OF DRAWINGS

FIG. 1 A schematic diagram showing a preparation protocol of DFAT cells in examples.

FIG. 2 A microscopic image of the DFAT cells obtained in Example 1.

FIG. 3 A microscopic image of the DFAT cells obtained in Example 2.

FIG. 4 A graph showing the cell number of the DFAT cells obtained in Example 2.

FIG. 5 A diagram showing the results of confirming the expression of various markers of the DFAT cells subcultured in Example 2.

FIG. 6 A graph showing the results of examining the proliferative ability of the DFAT cells subcultured in Example 2.

FIG. 7 A microscopic image of the DFAT cells obtained in Example 3.

FIG. 8 A graph showing the cell number of the DFAT cells obtained in Example 3.

FIG. 9 A a graph showing the cell number of the DFAT cells obtained by being cultured in culture media with different HPL concentrations in Example 4.

FIG. 10 A microscopic image of the DAFT cells obtained in Example 5.

FIG. 11 A graph showing the cell number of the DFAT cells obtained in Example 5.

FIG. 12 A diagram showing the results of confirming the expression of various MSC-positive markers of the DFAT cells obtained in Example 5.

FIG. 13 A diagram showing the results of confirming the expression of various MSC-negative markers of the DFAT cells obtained in Example 5.

FIG. 14 A graph showing the cell number of the DFAT cells subcultured in Example 5.

FIG. 15 A diagram showing the results of confirming the expression of various MSC-positive markers of the DFAT cells subcultured in Example 5.

FIG. 16 A diagram showing the results of confirming the expression of various MSC-negative markers of the DFAT cells subcultured in Example 5.

### DESCRIPTION OF EMBODIMENTS

The term “comprise” denotes that a constituent element other than the constituent element as a target may be included. The term “consist of” denotes that a constituent element other than the constituent element as a target is not included. The term “consist essentially of” denotes that a constituent element other than the constituent element as a target is not included in an aspect (for example, an aspect in which the effects of the invention are lost) that exhibits a special function. In the present specification, the description of “comprise” includes the aspect of “consist of” and the aspect of “consist essentially of”.

The cells described in the present specification can be isolated cells. The term “isolated” denotes a natural state or a state of separation from other components. The “isolated” cells do not substantially include other components. The expression “do not substantially include other components” denotes that the amount of other components included in the isolated component is negligible. The amount of other components included in the isolated component is, for example, 10% by mass or less, 5% by mass or less, 4% by mass or less, 3% by mass or less, 2% by mass or less, 1% by mass or less, 0.5% by mass or less, or 0.1% by mass or less. The human mature adipocytes and the human dedifferentiated fat cells described in the present specification can be isolated human mature adipocytes and isolated human dedifferentiated fat cells.

### Method for Producing Human DFAT Cells

### First Embodiment

A method for producing human DFAT cells of the present embodiment (hereinafter, also simply referred to as “production method of the present embodiment”) includes the following step:
a step (hereinafter, also referred to as “step 2”) of culturing the human mature adipocytes using a culture medium containing HPL.

The present inventors have searched for a component that can be replaced with FBS, which is an essential component during the induction of human DFAT cells from human mature adipocytes, and found HPL as a component capable of inducing human DFAT cells from human mature adipocytes. As shown in the examples described below, in a case of using the HPL, the induction of the DFAT cells can be carried out at a lower concentration than that in a case of using the FBS, the proliferation rate of the induced DFAT cells is excellent, and thus a larger amount of DFAT cells can be produced in a shorter period of time.

It is preferable that the culture medium used in the production method according to the present embodiment does not contain a component derived from an animal other than a human. For example, it is preferable that the culture medium be xeno-free. In general, the term “xeno-free” is used to denote that a culture medium does not contain a component derived from different species, does not contain a component derived from an animal, or does not contain a component derived from a non-human, and has a grade required in the development of regenerative medical materials. In the present specification, “xeno-free” means that the culture medium does not include a component derived from an animal other than a human. Examples of the component derived from an animal other than a human referred to herein include blood serum, blood components, and proteins derived from an animal other than a human. Examples of the blood serum derived from an animal other than a human include adult bovine serum, calf serum, newborn calf serum, and fetal bovine serum.

It is preferable that the culture medium used in the production method according to the present embodiment does not contain a component derived from an animal other than a human. Specific examples of the culture medium used in the production method according to the present embodiment include a culture medium containing HPL; a culture medium containing HLP and not containing FBS; a culture medium containing HLP and not containing proteins of an animal other than a human; a culture medium containing HLP and not containing blood serum of an animal other than a human; a culture medium containing HLP and not containing a blood component of an animal other than a human; a culture medium containing HLP and not containing a component of an animal other than a human; and a culture medium containing HLP and being xeno-free.

The human mature adipocytes used in the production method according to the present embodiment can be obtained as follows. The production method according to the present embodiment may include a step of obtaining human mature adipocytes (hereinafter, also referred to as “step 1”) before the step 2.

### <Step 1>

In the step 1, human mature adipocytes are obtained from human adipose tissues.

The human adipose tissues can be used by collecting tissues from, for example, the subcutaneous tissues, the internal organs (pericardium or mesentery), the bone marrow, the infrapatellar fat pad, or the buccal fat pad. Since the invasiveness associated with the collection is low and a large number of mature adipocytes of about 10⁶ cells can be obtained from about 1 g of excised adipose tissues or about 1 mL of suctioned adipose tissues, it is preferable that the human adipose tissues be collected from the subcutaneous tissues.

The human mature adipocytes can be obtained by decomposing the extracellular matrix of human adipose tissues. For example, an extracellular matrix-decomposing enzyme can be used for decomposition of the extracellular matrix. Specific examples of such an enzyme include collagenase. For example, the human mature adipocytes can be obtained by treating human adipose tissues with collagenase. The concentration of the collagenase used for the treatment of the human adipose tissues is preferably 0.01 w/v % or greater and 0.1 w/v % or less and more preferably 0.02 w/v % or greater and 0.05 w/v % or less.

In the step 1, the adipose tissues can be treated with the collagenase by adding a basal culture medium containing collagenase or water for injection containing collagenase to the adipose tissues.

Examples of the basal culture medium include Dulbecco's modified Eagle's medium (DMEM), Minimum Essential Medium (MEM), knockout-DMEM (KO-DMEM), Glasgow's MEM (G-MEM), Basal Medium Eagle (BME), DMEM/Ham F12, Advanced DMEM/Ham F12, Iscove's modified Dulbecco's medium, Ham F-10, Ham F-12, the 199 culture medium, and the RPMI 1640 culture medium, but the present invention is not limited thereto.

It is also preferable that the culture medium used in the step 1 does not contain a component derived from an animal other than a human.

Alternatively, in the step 1, the treatment can also be performed by adding the adipose tissues to a solution obtained by diluting Liberase MNP-S GMP Grade (manufactured by Roche) which is a xeno-free collagenase-containing digestive enzyme with water for injection such that the collagenase concentration is in the above-described ranges.

In the step 1, the collagenase treatment can be carried out, for example, at a temperature of 20° C. or higher and 40° C. or lower for a treatment time of 10 minutes or longer and 2 hours or shorter.

In the step 1, the cell suspension obtained by the collagenase treatment of the adipose tissues may be filtered. As a result, the mature adipocytes can be isolated as a single fraction consisting of unicellular adipocytes. The pore size of the membrane used for the filtration can be, for example, 100 μm or greater and 500 μm or less. The filtration can be performed once or a plurality of times, that is, twice or more. The pore size of the membrane used in a case where the filtration is performed a plurality of times may be the same or different for each filtration. From the viewpoint of more efficiently recovering mature adipocytes, it is preferable to use a combination of membranes having different pore sizes and more preferable that the pore size of the membrane used in the (n+1)-th filtration be smaller than the pore size of the membrane used in the n-th filtration (n represents an integer of 1 or greater).

### <Step 2>

In the step 2, the human mature adipocytes are cultured using a culture medium containing HPL. In this manner, the human DFAT cells can be obtained.

A commercially available product may be used as the HPL contained in the culture medium used in the step 2, or the HPL may be prepared from human platelets by a known method.

Examples of the commercially available HPL include UltraGRO™-PURE (manufactured by AventaCell Biomedical), Human Platelet Lysate (manufactured by StemCell Technologies), PLTGold™ Human Platelet Lysate (manufactured by Sigma Ardrich), and PLUS™ Cell Culture Supplement (manufactured by COMPASS Biomedical).

In addition, for example, the HPL can be obtained by freezing and thawing a human platelet lysate and removing a precipitate by centrifugation using the human platelet lysate as a raw material (see, for example, Reference 1: “Hemeda H et al., “Evaluation of human platelet lysate versus fetal bovine serum for culture of mesenchymal stromal cells.”, Cytotherapy, Vol. 16, Issue 2, pp. 170-180, 2014.”; Reference 2: “Astori G et al., “Platelet lysate as a substitute for animal serum for the ex-vivo expansion of mesenchymal stem/stromal cells: present and future.”, Stem Cell Res Ther., Vol. 7, Article No. 93, 2016.”; and Reference 3: “Burnouf T et al., “Human platelet lysate: Replacing fetal bovine serum as a gold standard for human cell propagation?”, Biomaterials, Vol. 76 pp. 371-387, 2016.”). Alternatively, the HPL can be obtained by centrifuging human platelet-rich plasma (hereinafter, also referred to as “PRP”) using the PRP as a raw material to obtain a supernatant. The HPL obtained by the above-described method may be used after performing a component analysis and confirming safety.

The culture medium used in the step 2 can be prepared by adding the HPL to a basal culture medium.

The concentration of HPL in the culture medium is preferably 0.5 w/v % or greater and 20.0 w/v % or less, more preferably 1.0 w/v % or greater and 15.0 w/v % or less, still more preferably 2.5 w/v % or greater and 15.0 w/v % or less, and particularly preferably 5.0 w/v % or greater and 15.0 w/v % or less. In a case where the concentration of HPL in the culture medium is greater than or equal to the above-described lower limits and less than or equal to the above-described upper limits, the induction of the human DFAT cells from the human mature adipocytes can be more efficiently performed. Meanwhile, in the case where the concentration of HPL in the culture medium is greater than or equal to the above-described lower limits and less than or equal to the above-described upper limits, the cell proliferative ability of the induced DFAT cells is more excellent.

As the basal culture medium, a basal culture medium used for culturing animal cells can be used. The basal culture medium is preferably a synthetic culture medium. The term “synthetic culture medium” denotes a culture medium of which the chemical composition is clear. The synthetic culture medium can be prepared by mixing purified chemical substances. Examples of the basal culture medium include Dulbecco's modified Eagle's medium (DMEM), Minimum Essential Media (MEM), knockout-DMEM (KO-DMEM), Glasgow's MEM (G-MEM), Basal Medium Eagle (BME), DMEM/Ham F12, Advanced DMEM/Ham F12, Iscove's modified Dulbecco's medium, Ham F-10, Ham F-12, the 199 culture medium, RPMI 1640 culture medium, CSTI303-MSC (manufactured by Cell Science & Technology Institute, Inc.), and CiMS-BM (manufactured by Cell Science & Technology Institute, Inc.), but the present invention is not limited thereto. In a case where a commercially available basal culture medium used for culturing MSC such as CiMS-BM is used, the cell proliferative ability of the induced DFAT cells is more excellent.

The culture medium used in the step 2 may contain other components in addition to the basal culture medium and HPL. For example, the culture medium can further contain an amino acid such as a nonessential amino acid or L-glutamine; an antibiotic substance such as penicillin, streptomycin, or gentamicin; a hormone such as insulin; and a growth factor such as basic fibroblast growth factor (bFGF). It is preferable that the culture medium used in the step 2 does not contain a component derived from an animal other than a human.

The details of the step 2 will be described below.

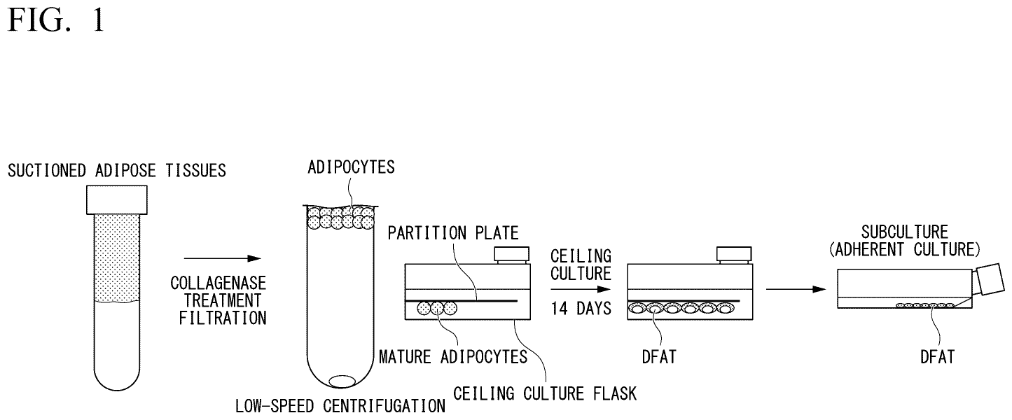

First, the human mature adipocytes obtained in the step 1 are seeded in a culture container. The culture container is not particularly limited as long as the container has a shape in which the mature adipocytes can adhere. The culture container is preferably a container that can be ceiling-cultured and preferably a lidded container having a flat bottom surface and a flat ceiling surface. As the culture container, for example, a tissue culture flask, a culture dish, or the like can be used. Alternatively, the culture container may include a partition plate that partitions a space in the culture container in a horizontal direction. The partition plate is disposed in the culture container such that the partition plate faces the bottom surface. For example, a ceiling culture flask as shown in FIG. 1 can also be used. As the ceiling culture flask, a culture container or the like described in PCT International Publication No. WO2017/078007A (Reference 4) can be used. As the ceiling culture flask, for example, a commercially available product (manufactured by SANPLATEC Corp.) or the like may be used.

Next, a culture container is filled with the culture medium containing the HPL. It is preferable that the culture medium be placed in the culture container in an amount set such that the human mature adipocytes in the culture container are in contact with the ceiling surface of the culture container. For example, in a case where a typical tissue culture flask is used as the culture container, the culture container can be filled with the culture medium in an amount of 90% by volume or greater and preferably 95% by volume or greater with respect to the volume of the culture container. The upper limit of the amount of the culture medium may be an amount set such that a part of the vapor phase in the culture container can remain, and can be set to less than 100% by volume and preferably 99% by volume or less with respect to the volume of the culture container. Thereafter, the human mature adipocytes are cultured in the culture container. It is preferable that the human mature adipocytes be cultured by ceiling culture using plastics as a scaffold material, but can also be cultured in a collagen gel or with a nonwoven fabric, and the present invention is not limited thereto.

In a case where a ceiling culture flask as shown in FIG. 1 is used, the suspension of the human mature adipocytes is seeded on the lower surface of the partition plate in the culture flask with a pipette or the like, and then the culture medium is added from the upper surface of the partition plate such that the amount of the culture medium is greater than or equal to the partition plate. Thereafter, the human mature adipocytes are cultured. It is preferable that the human mature adipocytes be cultured by ceiling culture using the partition plate as a ceiling surface.

Typical culture conditions used for culturing animal cells can be used as the culture conditions. The temperature condition may be, for example, 20° C. or higher and 40° C. or lower (preferably 37° C.). The carbon dioxide concentration may be, for example, 5 v/v % CO₂. The culture can be performed, for example, in a culture apparatus in a 5 v/v % CO₂ atmosphere. The seeding cell density of the human mature adipocytes is preferably 100 cells/mm² or greater and 400 cells/mm² or less. The culture period can be set to 3 days or longer and 20 days or shorter and preferably 7 days or longer and 14 days or shorter. It is preferable that the culture medium be exchanged every 5 days or longer and 10 days or shorter where possible.

As the time elapses from the start of the culture, many cells showing a fibroblast-like form without fat droplets, which are divided and derived from the human mature adipocytes having large fat droplets adhered to the ceiling surface (or the partition plate) of the culture container, are observed. The cells having a fibroblast-like form are human DFAT cells.

The DFAT cells are cells having MSC-like characteristics, which was produced by the present inventors, and are positive for CD73, CD90, and CD105, which are MSC-positive markers and negative for CD31, CD45, and HLA-DR, which are MSC-negative markers. These markers are cell surface antigens, and can be detected by, for example, flow cytometry or immunostaining. Therefore, the purity of the DFAT cells can be confirmed by detecting these cell surface antigens. Specifically, for example, in the flow cytometry using a fluorescence-labeled antibody that specifically binds to a specific cell surface antigen, in a case where cells emitting stronger fluorescence than in a negative control (isotype control) are detected, the cells are determined to be “positive” for the cell surface antigen. As the fluorescence-labeled antibody, any antibody known in the technical field can be used, and examples thereof include an antibody labeled with fluorescein isothiocyanate (FITC), phycoerythrin (PE), or allophycocyanin (APC), and the present invention is not limited thereto. In the immunostaining using a fluorescence-labeled antibody that specifically binds to a specific cell surface antigen, in a case where stained cells or cells emitting fluorescence are observed under a microscope, the cells are determined to be “positive” for the cell surface antigen.

In the production method according to the present embodiment, the step 1 and the step 2 can also be performed, for example, with reference to the method for preparing the DFAT cells described in Japanese Patent No. 5055611 (Reference 5), Japanese Patent No. 5055613 (Reference 6), Japanese Patent No. 5991687 (Reference 7), and the like.

### <Other Steps>

The production method according to the present embodiment may include other steps. Examples of the other steps include a step of subculturing human DFAT cells, a step of recovering human DFAT cells, and a step of cleaning human DFAT cells.

The step of subculturing the human DFAT cells can be carried out by a known method. The DFAT cells obtained after the above-described step 2 can be used for a desired application after being subcultured at least once. Examples of the culture medium used in the subculture include the same culture media as those used in the above-described step 2. In the subculture, it is preferable to perform adherent culture (adhesion culture) as shown in FIG. 1. The adherent culture can be performed, for example, using a culture container provided with an adhesive layer on the bottom surface. The adhesive layer can be formed on the bottom surface of the culture container, for example, using an extracellular matrix such as laminin, fibronectin, I-type collagen, and gelatin. Examples of the culture container include the same culture containers as described above. The culture conditions such as the culture temperature and the carbon dioxide concentration can be set to be the same as those in the above-described step 2. The subculture interval can be set to 3 days or longer and 20 days or shorter and preferably 4 days or longer and 14 days or shorter.

The step of recovering the human DFAT cells can be performed by a known method after the step 2 or the subculturing step. The human DFAT cells after the step 2 can be recovered by releasing the human DFAT cells attached to the ceiling of the culture container or the partition plate with an enzyme such as trypsin, and filtering or centrifuging the human DFAT cells. The human DFAT cells after the subculture can be recovered by releasing the human DFAT cells attached to the bottom surface of the culture container with an enzyme such as trypsin, and filtering or centrifuging the human DFAT cells.

The step of cleaning the human DFAT cells can be performed using a known method after the step of recovering the human DFAT cells. The cleaning of the human DFAT cells can be performed on the human DFAT cells recovered by the step of recovering the human DFAT cells. The cleaning of the human DFAT cells can be performed, for example, by repeating (a) suspension of the human DFAT cells in a buffer solution or the like and (b) recovery of the human DFAT cells from the suspension by filtration or centrifugation.

### Second Embodiment

A method for producing the human DFAT cells according to the present embodiment includes the following step:
a step (hereinafter, also referred to as “step A”) of culturing human DFAT cells using a culture medium containing HPL.

### <Step A>

The culture medium used in the step A is a culture medium containing HPL. It is preferable that the culture medium used in the step A does not contain a component derived from a human or an animal and is xeno-free. Examples of the culture medium used in the step A include the same culture media as those used in the above-described step 2.

The human DFAT cells used in the step A can be produced by the above-described method for producing human DFAT cells according to the first embodiment. The culture in the step A is preferably adhesion culture. The culture in the step A can be carried out in the same manner as in the subculturing step described in the first embodiment.

### <Other Steps>

The method for producing human DFAT cells according to the present embodiment may include other steps in addition to the step A. Examples of the other steps performed before the step A include the step 1 and the step 2 described above. Examples of the other steps performed after the step A include a step of recovering the above-described human DFAT cells and a step of cleaning the human DFAT cells.

As shown in the examples described below, the induction efficiency of the human DFAT cells from the human mature adipocytes can be improved by using a culture medium containing HPL, and the proliferation rate of the produced human DFAT cells can be improved. Therefore, a large amount of human DFAT cells can be efficiently produced in a short period of time.

### Culture Medium for Producing Human DFAT Cells from Human Mature Adipocytes

The culture medium of the present embodiment is a culture medium for producing human DFAT cells from human mature adipocytes, and contains HPL.

It is preferable that the culture medium according to the present embodiment does not contain a component derived from an animal other than a human and is xeno-free.

The culture medium according to the present embodiment can be prepared by adding HPL to a basal culture medium and mixing the HPL into the basal culture medium.

The specific composition of the culture medium according to the present embodiment is as described in the step 2 of “method for producing human DFAT cells” described above.

### Composition

The composition according to the present embodiment contains human DFAT cells and HPL.

The composition according to the present embodiment can be obtained by the method for producing human DFAT cells according to the first embodiment or the second embodiment described above.

The composition according to the present embodiment may be a culture solution containing the human DFAT cells obtained after the above-described step 2. The culture solution contains HPL, which is a culture medium component, and the human DFAT cells induced from the human mature adipocytes.

The composition according to the present embodiment may be a culture solution containing the human DFAT cells obtained after the above-described step A. The culture solution contains HPL, which is a culture medium component, and proliferated human DFAT cells.

The composition according to the present embodiment may be the human DFAT cells obtained after the step of recovering the human DFAT cells. The HPL, which is a culture medium component, remains and is attached to the human DFAT cell.

The composition according to the present embodiment may be human DFAT cells obtained after the step of cleaning the human DFAT cells. Even in a case where the DFAT cells are repeatedly cleaned, the components of the culture medium are difficult to completely remove. Therefore, the HPL, which is a culture medium component, remains and is attached to the human DFAT cells.

In a case where the composition according to the present embodiment is the human DFAT cells (hereinafter, also referred to as “human DFAT cell purified product”) obtained after the step of cleaning the human DFAT cells, the human DFAT cell purified product can be used as a regenerative medical pharmaceutical product. The proportion of the HPL in the human DFAT cell purified product is less than 1/10¹² with respect to the total mass of the human DFAT cell purified product.

### EXAMPLES

Hereinafter, the present invention will be described based on the examples, but the present invention is not limited to the following examples.

### Example 1

### (Selection of FBS Substitute for Preparing Human DFAT Cells)

### 1. Induction of Human DFAT Cells from Human Mature Adipocytes

It was confirmed whether human DFAT cells could be prepared in the same manner as that for FBS by using an FBS substitute used for culturing MSC.

### (1) Preparation of Human Adipose Tissues

Adipose tissues were collected from a subject (58 years old, female) by adipose suction with the consent of the subject. 2 mL of the obtained adipose tissues were subjected to a collagenase treatment (37° C., 30 minutes). As the collagenase, Liberase MNP-S GMP Grade (manufactured by Roche) was used. Next, the extracellular matrix components were removed from the adipose tissues using a 100 μm cell strainer (manufactured by Corning), and then the obtained adipose tissues were filtered and centrifuged at a low speed, thereby obtaining a fraction of mature adipocytes (floating phase).

### (2) Induction of DFAT Cells

The cells were cultured in order to induce the cells into DFAT cells using a culture medium having the following composition. As an FBS substitute, a human platelet lysate (UltraGRO™-PURE (manufactured by AventaCell BioMedical) (hereinafter, “UltraGRO”) and a basal culture medium animal-free additive for human mesenchymal stem cells (CiMS-sAF (manufactured by Cell Science & Technology Institute, Inc.) (hereinafter, “sAF”) were used. As a basal culture medium, CSTI303-MSC (manufactured by Cell Science & Technology Institute, Inc.) (hereinafter, “CSTI”) and CiMS-BM (manufactured by Cell Science & Technology Institute, Inc.) (hereinafter, “CiMS”) were used. The culture medium was prepared by adding FBS or a serum substitute to the basal culture medium. The culture medium was prepared in advance to have any of the following 1) to 4) compositions. As the culture container, a ceiling culture flask (manufactured by Sanplatec Corp.) shown in FIG. 1 was used.
1) 5.0 w/v % UltraGRO-containing CSTI culture medium
2) 5.0 w/v % of UltraGRO-containing CiMS culture medium
3) 2.0 w/v % of sAF-containing CiMS culture medium
4) 20.0 w/v % FBS-containing CSTI culture medium

Specifically, the cell culture was performed by the following operation.

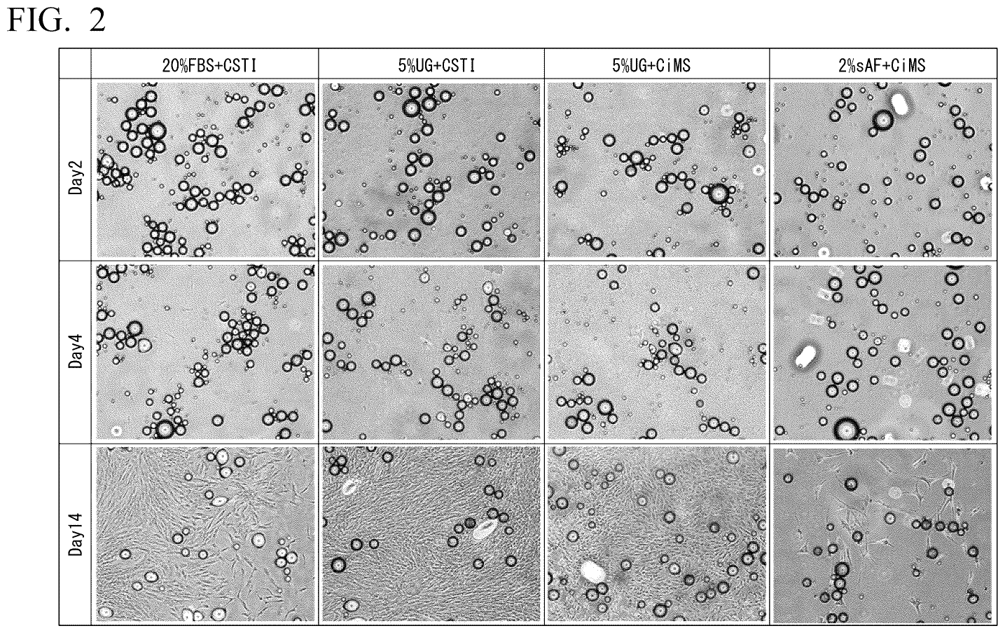

First, 100 μL of the fraction (floating phase) of mature adipocytes obtained in the section “(1) Preparation of Human Adipose Tissues” described above was suspended in 30 mL of each culture medium of the items 1) to 4) described above, and seeded on a cell adhesion surface of a lower surface of a partition plate of the ceiling culture flask. Next, 20 mL of each culture medium was added to an upper portion of the partition plate of the ceiling culture flask. Next, ceiling culture was performed for 14 days in an atmosphere of 37° C. and 5 v/v % CO₂. The culture medium was exchanged 7 days after the start of the ceiling culture. 2 days, 4 days, and 14 days after the start of the ceiling culture, the cells were observed with a phase contrast microscope. The results are shown in FIG. 2.

As shown in FIG. 2, in a case where the 5.0 w/v % UltraGRO-containing CSTI culture medium or the 5.0 w/v % UltraGRO-containing CIMS culture medium was used, the DFAT cells could be produced by the ceiling culture in the same manner as in a case where the 20.0 w/v % FBS-containing CSTI culture medium was used. However, in a case where the 2.0 w/v % sAF-containing CiMS culture medium was used, the DFAT cells could be produced, but there was a tendency that the cell proliferative ability was degraded as compared with a case where UltraGRO was used. As shown in the results described above, a human platelet lysate (hereinafter, “HPL”) was selected as an FBS substitute for preparing human DFAT cells.

### Example 2

### (Comparative Test Between HPL and FBS)

### 1. Induction of Human DFAT Cells from Human Mature Adipocytes

It was confirmed whether human DFAT cells could be prepared using HPL as a substitute for FBS.

### (1) Preparation of Human Adipose Tissues

Adipose tissues were collected from subjects (donor 1: 60-year-old female, donor 2: 30-year-old female, donor 3: 43-year-old female, donor 4: 56-year-old male) by adipose suction with the consent of the subjects. 2 mL of the obtained adipose tissues were subjected to a collagenase treatment (37° C., 30 minutes). As the collagenase, Liberase MNP-S GMP Grade (manufactured by Roche) was used. Next, the extracellular matrix components were removed from the adipose tissues using a 100 μm cell strainer (manufactured by Corning), and then the obtained adipose tissues were filtered and centrifuged at a low speed, thereby obtaining a fraction of mature adipocytes (floating phase).

### (2) Induction of DFAT Cells

The mature adipocytes were cultured in order to induce the cells into DFAT cells using a culture medium having any one of the following 1) to 3) compositions. As HPL, UltraGRO™-PURE (manufactured by AventaCell BioMedical) was used. CSTI303-MSC (manufactured by Cell Science & Technology Institute, Inc.) was used as a basal culture medium. The culture medium was prepared by adding FBS or HPL to the basal culture medium. The culture medium was prepared in advance to have any of the following 1) to 4) compositions. As the culture container, a ceiling culture flask (manufactured by Sanplatec Corp.) shown in FIG. 1 was used.
1) 10.0 w/v % HPL-containing CSTI culture medium
2) 5.0 w/v % HPL-containing CSTI culture medium
3) 20.0 w/v % FBS-containing CSTI culture medium

Specifically, the cell culture was performed by the following operation.

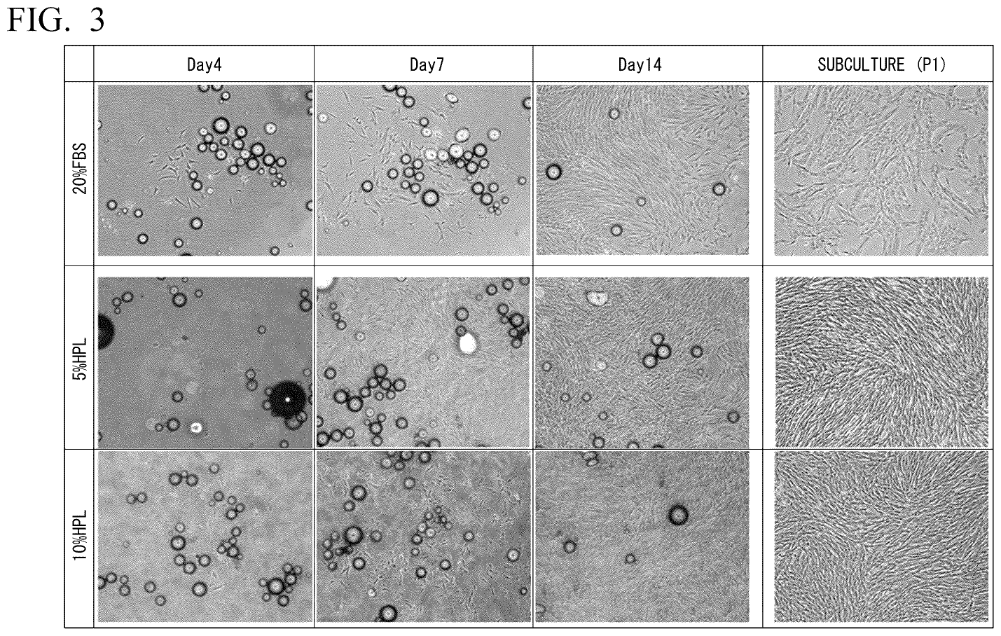

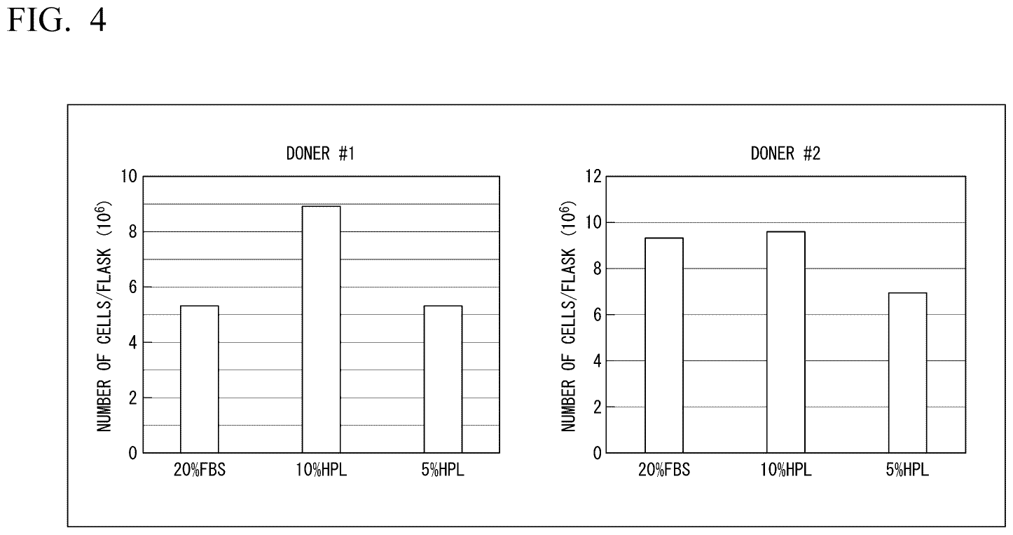

First, 100 μL of the fraction (floating phase) of mature adipocytes obtained in the section “(1) Preparation of human adipose tissues” described above was suspended in 30 mL of each culture medium of the items 1) to 3) described above, and seeded on a cell adhesion surface of a lower surface of a partition plate of the ceiling culture flask. Next, 20 mL of each culture medium was added to an upper portion of the partition plate of the ceiling culture flask. Next, ceiling culture was performed for 14 days in an atmosphere of 37° C. and 5 v/v % CO₂. The culture medium was exchanged 7 days after the start of the ceiling culture. 4 days, 7 days, and 14 days after the start of the ceiling culture, the cells were observed with a phase contrast microscope. Next, the cells were subjected to a trypsin treatment 14 days after the start of the ceiling culture, and the number of the obtained DFAT cells was measured. Next, subculture (P1) of the obtained DFAT cells was carried out. The results are shown in FIGS. 3 and 4.

As shown in FIG. 3, in a case where the 5.0 w/v % HPL-containing culture medium and the 10.0 w/v % HPL-containing culture medium were used, the DFAT cells could be produced and subcultured by ceiling culture as in a case where the 20.0 w/v % FBS-containing culture medium was used.

As shown in FIG. 4, the cell number of the DFAT cells obtained 14 days after the ceiling culture was not significantly different between a case of using the 5.0 w/v % HPL-containing culture medium and the 10.0 w/v % HPL-containing culture medium and a case of using the 20.0 w/v % FBS-containing culture medium.

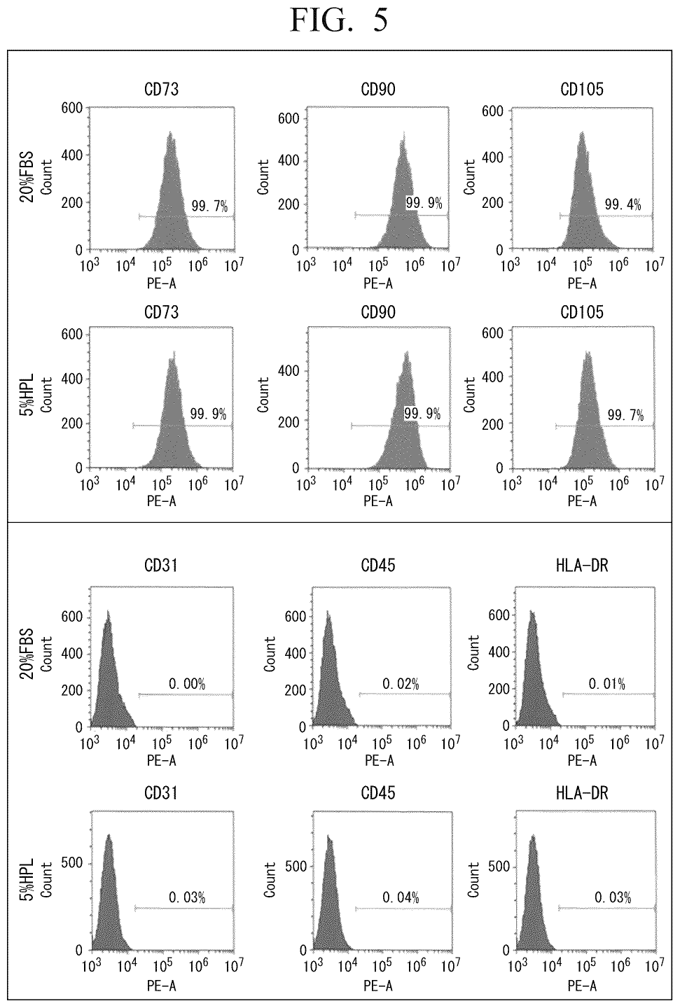

After the subculture was performed for a total of 21 days (every 7 days, 3 times; P1, P2, and P3), the cell surface antigens of the DFAT cells were analyzed by a flow cytometer. The confirmed cell surface antigens were CD73, CD90, and CD105, which are MSC-positive markers, and CD31, CD45, and HLA-DR, which are MSC-negative markers. As a representative example, the results of the DFAT cells (P1) that were subcultured using the 5.0 w/v % HPL-containing culture medium and the 20.0 w/v % FBS-containing culture medium are shown in FIG. 5.

As shown in FIG. 5, the cell surface antigen profile of the DFAT cells obtained using the 5.0 w/v % HPL-containing culture medium matched the cell surface antigen profile of the DFAT cells obtained using the 20.0 w/v % FBS-containing culture medium. Specifically, CD73, CD90, and CD105, which are MSC-positive markers, were positive in 90% or greater for any of the DFAT cells. Meanwhile, the positive rates of CD31,CD45, and HLA-DR, which are the MSC-negative markers, were less than 0.1% in any of the DFAT cells. The same results were obtained with the DFAT cells of P2 and P3.

### 2. Subculture of DFAT Cells

DFAT cells were obtained by the method described in the section “1. Induction of Human DFAT Cells from Human Mature Adipocytes Cells” described above using a 10 w/v % FBS-containing culture medium. The obtained DFAT cells were subcultured five times using a 10 w/v % FBS-containing culture medium, and the culture medium composition was examined.

The DFAT cells suspended in a 10 w/v % FBS-containing culture medium were seeded on a 24-well plate at a density of 2500 cells/well. Next, one day after the start of the culture, the culture medium was exchanged with any one of the following 1) to 3) culture media. As HPL, UltraGRO™-PURE (manufactured by AventaCell BioMedical) was used. CSTI303-MSC (manufactured by Cell Science & Technology Institute, Inc.) was used as a basal culture medium. The culture medium was prepared by adding FBS or HPL to the basal culture medium. The culture medium was prepared in advance to have any one of the following 1) to 3) compositions.
1) 10.0 w/v % HPL-containing CSTI culture medium
2) 5.0 w/v % HPL-containing CSTI culture medium
3) 10.0 w/v % FBS-containing CSTI culture medium

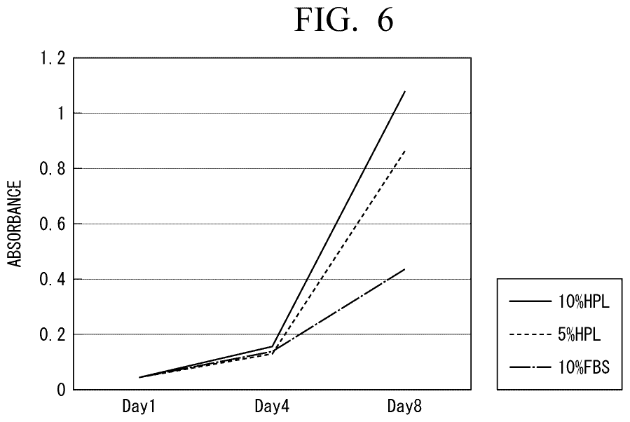

The proliferative ability of each cell was compared by performing WST-1 assay and measuring the absorbance before the culture medium exchange and 4 and 8 days after the start of the culture. The results are shown in FIG. 6.

As shown in FIG. 6, in a case where the 5.0 w/v % HPL-containing culture medium and the 10.0 w/v % HPL-containing culture medium were used, there was a tendency that the cell proliferative ability was more excellent than that in a case where the 10.0 w/v % FBS-containing culture medium was used.

### Example 3

### (Comparative Test of Commercially Available HPL)

### 1. Induction of Human DFAT Cells from Human Mature Adipocytes

It was examined whether the DFAT cells could be prepared by using various commercially available products of HPL.

### (1) Preparation of Human Adipose Tissues

Adipose tissues were collected from a subject (38 years old, female) by Adipose suction with the consent of the subject. 2 mL of the obtained adipose tissues were subjected to a collagenase treatment (37° C., 30 minutes). As the collagenase, Liberase MNP-S GMP Grade (manufactured by Roche) was used. Next, the extracellular matrix components were removed from the adipose tissues using a 100 μm cell strainer (manufactured by Corning), and then the obtained adipose tissues were filtered and centrifuged at a low speed, thereby obtaining a fraction of mature adipocytes (floating phase).

### (2) Induction of DFAT Cells

The cells were cultured in order to induce the cells into DFAT cells using a culture medium having any of the following 1) to 4) compositions. As HPL, UltraGRO™-PURE (manufactured by AventaCell Biomedical), Human Platelet Lysate (manufactured by StemCell Technologies), and PLTGold™ Human Platelet Lysate (manufactured by Sigma Ardrich) were used. CSTI303-MSC (manufactured by Cell Science & Technology Institute, Inc.) was used as a basal culture medium. The culture medium was prepared by adding FBS or HPL to the basal culture medium. The culture medium was prepared in advance to have any of the following 1) to 4) compositions. As the culture container, a ceiling culture flask (manufactured by Sanplatec Corp.) shown in FIG. 1 was used.
1) 20.0 w/v % FBS-containing CSTI culture medium
2) 5.0 w/v % UltraGRO™-PURE (manufactured by AventaCell BioMedical)-containing CSTI culture medium
3) 5.0 w/v % of Human Platelet Lysate (manufactured by StemCell Technologies)-containing CSTI culture medium
4) 5.0 w/v % PLTGold™ Human Platelet Lysate (manufactured by Sigma Ardrich)-containing CSTI culture medium

Specifically, the cell culture was performed by the following operation.

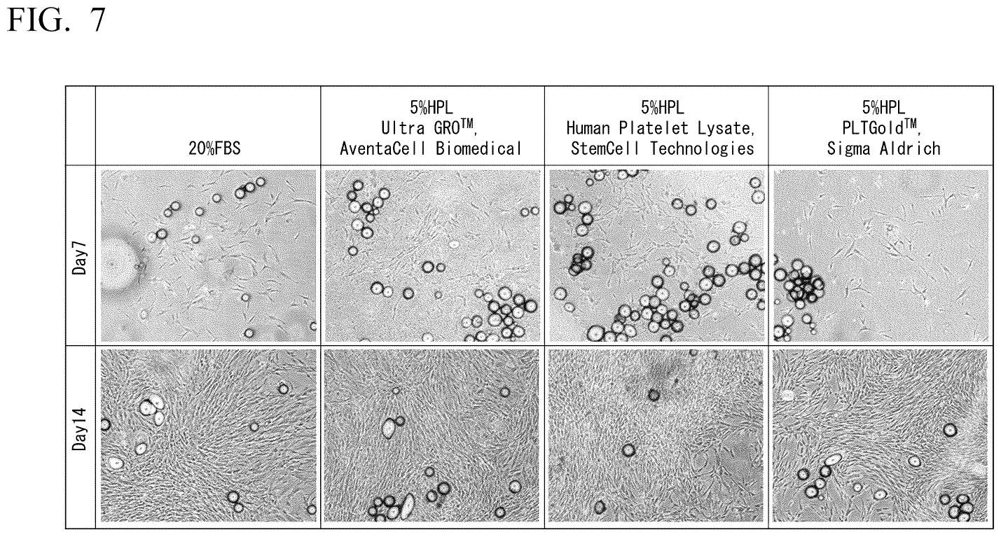

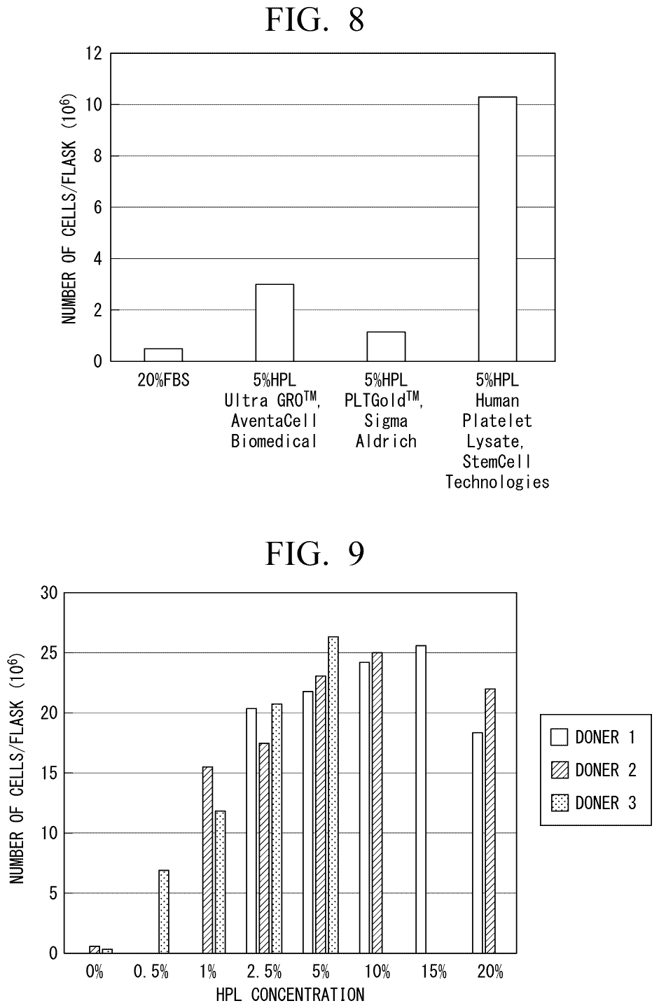

First, 100 μL of the fraction (floating phase) of mature adipocytes obtained in the section “(1) Preparation of Human Adipose Tissues” described above was suspended in 30 mL of each culture medium of the items 1) to 4) described above, and seeded on a cell adhesion surface of a lower surface of a partition plate of the ceiling culture flask. Next, 20 mL of each culture medium was added to an upper portion of the partition plate of the ceiling culture flask. Next, ceiling culture was performed for 14 days in an atmosphere of 37° C. and 5 v/v % CO₂. The culture medium was exchanged 7 days after the start of the ceiling culture. The cells were observed with a phase contrast microscope 7 days and 14 days after the start of the ceiling culture. Next, the cells were subjected to a trypsin treatment 14 days after the start of the ceiling culture, and the number of the obtained DFAT cells was measured. The results are shown in FIGS. 7 and 8

As shown in FIG. 7, even in a case where any of the 5.0 w/v % HPL-containing culture media was used, the DFAT cells could be produced by the ceiling culture as in a case where the 20.0 w/v % FBS-containing culture medium was used.

As shown in FIG. 8, even in a case where any of the 5.0 w/v % HPL-containing culture media was used, there was a tendency that the cell proliferative ability was more excellent than in a case where the 20.0 w/v % FBS-containing culture medium was used.

### Example 4

### (Examination of HPL Concentration)

### 1. Induction of Human DFAT Cells from Human Mature Adipocytes

DFAT cells were produced using HPLs having various concentrations, and the lower limit value of the HPL concentration at which the DFAT cells could be produced was examined.

### (1) Preparation of Human Adipose Tissues

Adipose tissues were collected from the subjects (donor 1: 51-year-old male, donor 2: 20-year-old female, donor 3: 37-year-old female) by Adipose suction with the consent of the subjects. 2 mL of the obtained adipose tissues were subjected to a collagenase treatment (37° C., 30 minutes). As the collagenase, Liberase MNP-S GMP Grade (manufactured by Roche) was used. Next, the extracellular matrix components were removed from the adipose tissues using a 100 μm cell strainer (manufactured by Corning), and then the obtained adipose tissues were filtered and centrifuged at a low speed, thereby obtaining a fraction of mature adipocytes (floating phase).

### (2) Induction of DFAT Cells

The mature adipocytes derived from each donor were cultured in order to induce the cells into the DFAT cells using a culture medium having the following composition. As HPL, UltraGRO™-PURE (manufactured by AventaCell BioMedical) was used. CiMS-BM (manufactured by Cell Science & Technology Institute, Inc.) was used as a basal culture medium. The culture medium was prepared by adding HPL to the basal culture medium. The composition of the culture medium used for the culture of the mature adipocytes derived from each donor is described below. As the culture container, a ceiling culture flask (manufactured by Sanplatec Corp.) shown in FIG. 1 was used.

Donor 1 (51-year-old male)
1) 2.5 w/v % HPL-containing CiMS culture medium
2) 5.0 w/v % HPL-containing CiMS culture medium
3) 10.0 w/v % HPL-containing CiMS culture medium
4) 15.0 w/v % HPL-containing CiMS culture medium
5) 20.0 w/v % HPL-containing CiMS culture medium

Donor 2 (20-year-old female)
1) 0.0 w/v % HPL-containing CiMS culture medium
2) 1.0 w/v % HPL-containing CiMS culture medium
3) 2.5 w/v % HPL-containing CiMS culture medium
4) 5.0 w/v % HPL-containing CiMS culture medium
5) 10.0 w/v % HPL-containing CiMS culture medium
6) 20.0 w/v % HPL-containing CiMS culture medium

Donor 3 (37-year-old female)
1) 0.0 w/v % HPL-containing CiMS culture medium
2) 0.5 w/v % HPL-containing CiMS culture medium
3) 1.0 w/v % HPL-containing CiMS culture medium
4) 2.5 w/v % HPL-containing CiMS culture medium
5) 5.0 w/v % HPL-containing CiMS culture medium

Specifically, the cell culture was performed by the following operation.

First, 100 μL of the fraction (floating phase) of mature adipocytes obtained in the section “(1) Preparation of human adipose tissues” described above was suspended in 30 mL of each culture medium, and seeded on a cell adhesion surface of a lower surface of a partition plate of the ceiling culture flask. Next, 20 mL of each culture medium was added to an upper portion of the partition plate of the ceiling culture flask. Next, ceiling culture was performed for 14 days in an atmosphere of 37° C. and 5 v/v % CO2. The culture medium was exchanged 7 days after the start of the ceiling culture. 14 days after the start of the ceiling culture, a trypsin treatment was performed, and the number of the obtained DFAT cells was measured. The results are shown in FIG. 9.

As shown in FIG. 9, even in a case where the 0.5 w/v % HPL-containing culture medium was used, the cell proliferative ability was also decreased, but the DFAT cells could be produced by the ceiling culture. Therefore, it was found that the use of the HPL-containing culture medium with 0.5 w/v % or greater enabled the production of the DFAT cells. From the viewpoint of the cell proliferative ability of the DAFT cells, it was considered that the optimum concentration of HPL was in a range of 5.0 to 15.0 w/v %.

### Example 5

### (Confirmation of Reproducibility)

It was confirmed whether the DFAT cells could be prepared with satisfactory reproducibility by a production method using a 5.0 w/v % HPL-containing xeno-free culture medium and a ceiling culture flask.

### (1) Preparation of Human Adipocytes

Adipose tissues were collected from the subjects (donor 1: 39-year-old male, donor 2: 43-year-old male, donor 3: 45-year-old female, donor 4: 28-year-old female, donor 5: 58-year-old female) by adipose suction with the consent of the subjects. 2 mL of the obtained adipose tissues were subjected to a collagenase treatment (37° C., 30minutes). As the collagenase, Liberase MNP-S GMP Grade (manufactured by Roche) was used. Next, the extracellular matrix components were removed using a 500 μm cell strainer and a 100 μm cell strainer (manufactured by pluriSelect). Thereafter, the obtained adipose tissues were filtered and centrifuged at a low speed, thereby obtaining a fraction of mature adipocytes (floating phase).

### (2) Induction of DFAT Cells

The mature adipocytes derived from each donor were cultured in order to induce the cells into the DFAT cells using a culture medium having the following composition. As HPL, UltraGRO™-PURE (manufactured by AventaCell BioMedical) was used. CiMS-BM (manufactured by Cell Science & Technology Institute, Inc.) was used as a basal culture medium. The culture medium was prepared in advance by adding HPL to the basal culture medium to obtain a 5.0 w/v % HPL-containing culture medium. As the culture container, a ceiling culture flask (manufactured by Sanplatec Corp.) shown in FIG. 1 was used. Specifically, the cell culture was performed by the following operation.

First, 100 μL of the fraction (floating phase) of mature adipocytes obtained in the section (1) described above was suspended in 30 ml of the culture medium, and seeded on a cell adhesion surface of a lower surface of a partition plate of the flask. Next, 20 mL of the culture medium was added to the upper portion of the partition plate of the flask. Next, ceiling culture was performed for 14 days in an atmosphere of 37° C. and 5 v/v % CO₂. The culture medium was exchanged 7 days after the start of the ceiling culture. The cells were observed with a phase contrast microscope 7 days and 14 days after the start of the ceiling culture. Next, a trypsin treatment was performed 14 days after the start of the ceiling culture, the number of the obtained DFAT cells was measured, and the cell surface antigen was measured using a flow cytometer.

Next, subculture (P1) of the obtained DFAT cells was carried out. 60 mL of the DFAT cells suspended in the culture medium were seeded in each of 8 T225 flasks (manufactured by Corning) at a density of 9×10⁵ cells/flask. The cells were observed with a phase contrast microscope 7 days after the subculture (21 days after the start of the ceiling culture). Thereafter, the trypsin treatment was performed, the number of the obtained DFAT cells was measured, and the cell surface antigen was measured using a flow cytometer.

### (3) Result

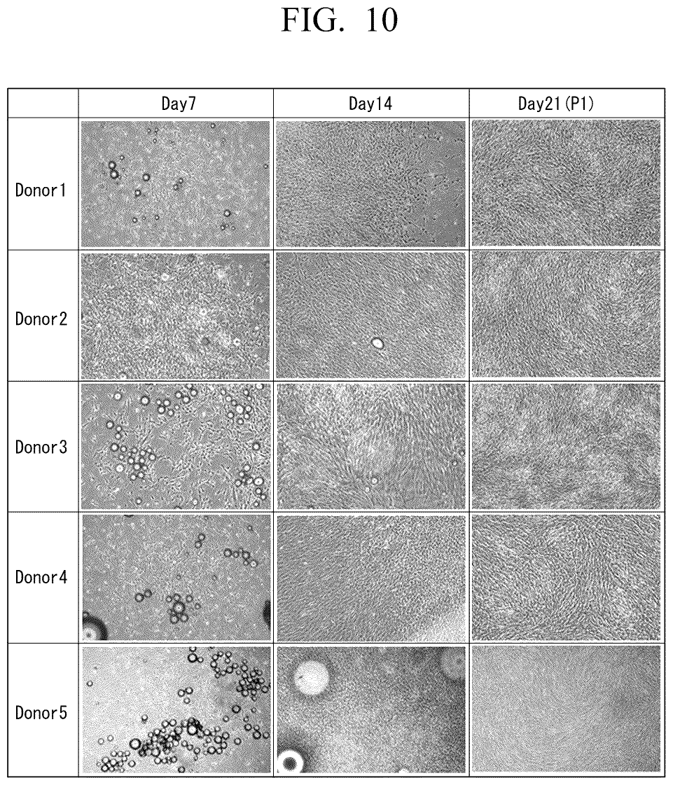

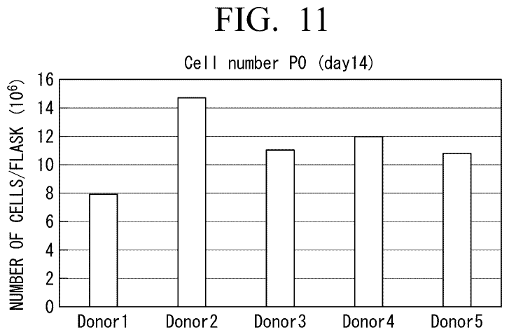

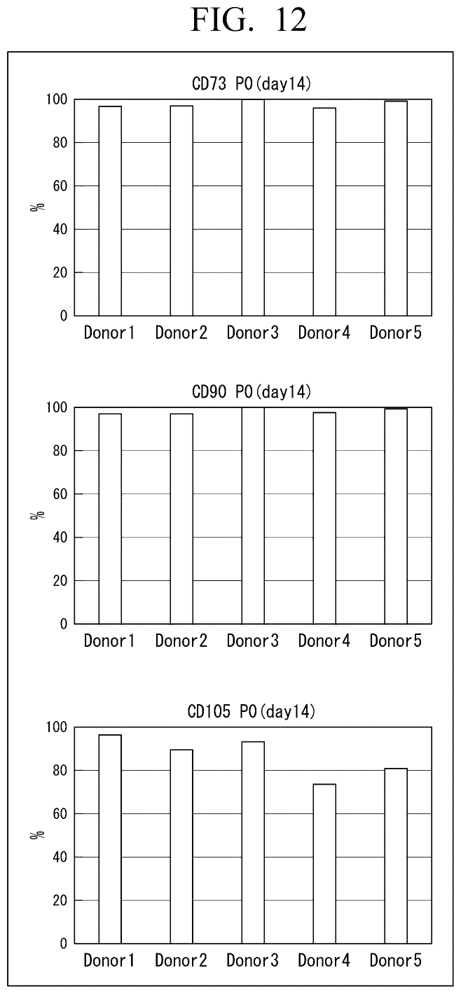

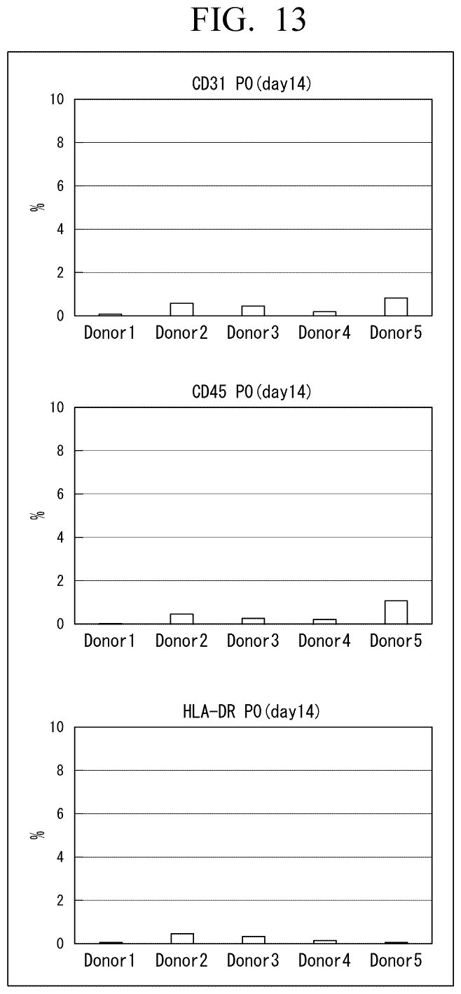

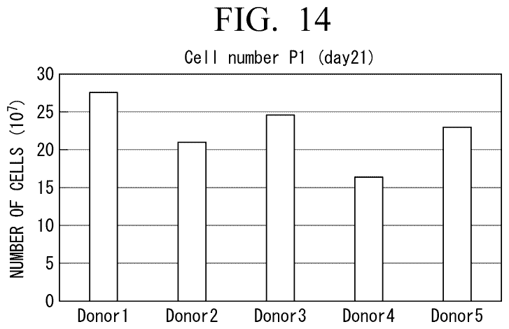

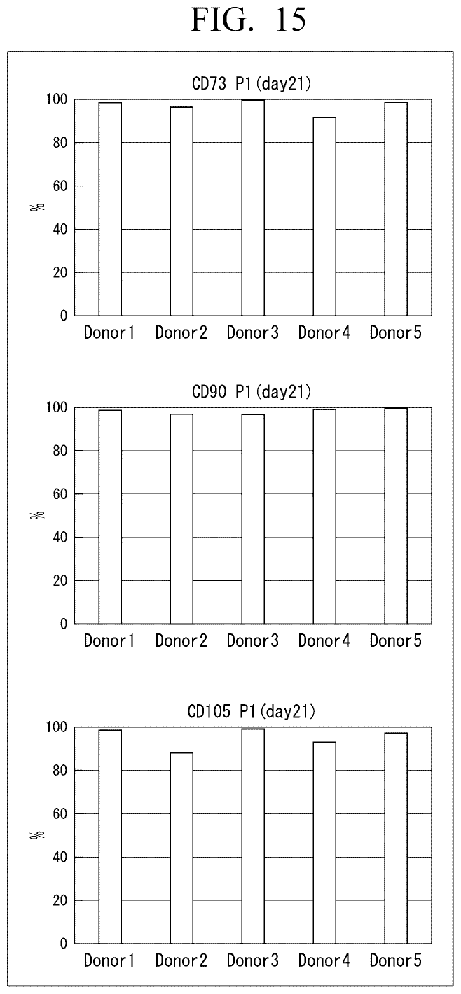

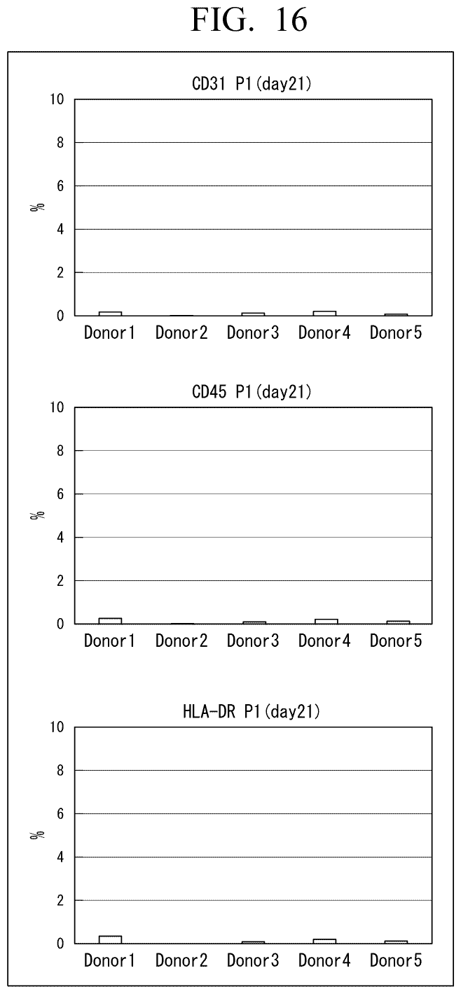

The results are shown in FIGS. 10 to 16. As shown in FIG. 10, in all the examples of the donors 1 to 5, the DFAT cells could be induced from mature adipocytes 7days after the start of the ceiling-culture by the production method using the 5.0 w/v % HPL-containing xeno-free culture medium and the ceiling culture flask. The proliferation of the DFAT cells was confirmed 14 days after the start of the ceiling culture as shown in FIG. 10, and the cell number of the DFAT cells 14 days after the start of the ceiling culture was 7×10⁶ cells/flask or greater as shown in FIG. 11. As shown in FIG. 12, the cell surface antigens of the DFAT cells 14 days after the start of the ceiling culture showed a positive rate of 70% or greater for CD73, CD90, and CD105, which are MSC-positive markers, Meanwhile, as shown in FIG. 13, the cell surface antigens showed a positive rate of less than 2% for CD31, CD45, and HLA-DR, which are the MSC-negative markers. In addition, as shown in FIG. 10, in all the examples of the donors 1to 5, the DFAT cells could be amplified by the subculture by the production method using the 5.0 w/v % HPL-containing xeno-free culture medium. As shown in FIG. 14, the number of DFAT cells 7 days after the subculture (21 days after the start of the ceiling culture) was 1.5×10⁸ cells or greater. As shown in FIG. 15, the cell surface antigens of the DFAT cells 7 days after the subculture (21 days after the start of the ceiling culture) showed a positive rate of 80% or greater for CD73, CD90, and CD105, which are MSC-positive markers. Meanwhile, as shown in FIG. 16, the cell surface antigens showed a positive rate of less than 1% for CD31, CD45, and HLA-DR, which are the MSC-negative markers. As shown in the above-described results, it was confirmed that the DFAT cells could be prepared with satisfactory reproducibility by the production method using the 5.0 w/v % HPL-containing xeno-free culture medium and the ceiling culture flask.

### INDUSTRIAL APPLICABILITY

According to the production method and the culture medium of the present embodiment, it is possible to produce human DFAT cells from mature human adipocytes without using FBS.

While preferred examples of the present invention have been described and illustrated above, it should be understood that these are examples of the present invention and are not to be considered as limiting. Additions, omissions, substitutions, and other modifications can be made without departing from the scope of the present invention. Accordingly, the present invention is not limited by the description above but by the scope of the appended claims.

1. A method for producing human dedifferentiated fat cells, comprising:
inducing human dedifferentiated fat cells from human mature adipocytes by culturing human mature adipocytes using a culture medium containing a human platelet lysate,
wherein the culture medium is a culture medium that does not contain a serum derived from an animal other than a human.

2. The method for producing human dedifferentiated fat cells according to claim 1,
wherein a concentration of the human platelet lysate in the culture medium is 0.5w/v % or greater and 20.0 w/v % or less.

3. The method for producing human dedifferentiated fat cells according to claim 2,
wherein the concentration of the human platelet lysate in the culture medium is 5.0 w/v % or greater and 15.0 w/v % or less.

4. The method for producing human dedifferentiated fat cells according to claim 1,
wherein the human mature adipocytes are cultured by ceiling culture.

5. The method for producing human dedifferentiated fat cells according to claim 1,
wherein the culture medium is a culture medium that does not contain a component derived from an animal other than a human.

6. A method for producing human dedifferentiated fat cells, comprising:
proliferating human dedifferentiated fat cells by culturing human dedifferentiated fat cells obtained by the method for producing human dedifferentiated fat cells according to claim 1 using a culture medium containing a human platelet lysate.

7. The method for producing human dedifferentiated fat cells according to claim 6,
wherein a concentration of the human platelet lysate in the culture medium is 0.5 w/v % or greater and 20.0 w/v % or less.

8. The method for producing human dedifferentiated fat cells according to claim 6,
wherein the human dedifferentiated fat cells are cultured by adhesion culture.

9.-11. (canceled)
